(19) United States
(12) Patent Application Publication
(10) Pub. No.: US 2004/0126421 A1
(43) Pub. Date: Jul. 1, 2004

(54) Liposomal system and method of using same

(76) Inventors: Mary Jo Turk, New York, NY (US); Jose Alejandro Guevara, Roosevelt Island, NY (US); Alan Houghton, New York, NY (US)

Correspondence Address: OPPEDAHL AND LARSON LLP, P O BOX 5068, DILLON, CO, 80435-5068, US

(21) Appl. No.: 10/680,062

(22) Filed: Oct. 6, 2003

Related U.S. Application Data
(60) Provisional application No. 60/416,194, filed on Oct. 4, 2002.

(51) Int. Cl. A61K 39/00; A61K 9/127

(52) U.S. Cl. 424/450; 424/185.1

(57) ABSTRACT

Liposomal system formed from a peptide having a ubiquitinatable region and an antigenic region, a pH-sensitive liposomal carrier; and a bilayer-associated adjuvant, such as MPL are useful as vaccine compositions. These compositions are surprisingly effective in inducing an in vivo immune response to the antigen corresponding to the antigenic region.

This application claims the benefit of U.S. Provisional Application Serial No. 60/416,194, filed Oct. 4, 2002, which is incorporated herein by reference.

### BACKGROUND OF THE INVENTION

This invention generally relates to a vaccine that will stimulate T-cell mediated immunity against an antigen. More specifically, it relates to vaccines that combine adjuvant-doped liposomes with peptide antigens that contain proteasome (ubiquitin) sorting sequences.

Among the challenges to providing effective vaccination to many disease conditions is the need to generate both a humoral or antibody response, and an effective CD8+ T cell response. Efforts have been reported to achieve this end with respect to HIV-1 and MUC 1 in Chang et al., Vaccine 17: 1540-1548 (1999) and Samuel et al., Int. J. Cancer 19: 295-302 (1998), respectively, using monophosphoryl Lipid A (MPL) adjuvanted liposomes. Both of these papers report induced antibody responses, but poor CD8+ T cell responses. Richards et al., Infection and Immunity 66: 2859-2865) report on similar composition with malaria antigen. U.S. Pat. No. 6,287,569, which is incorporated herein by reference, discloses a method for generating, in a patient, a cellular immune response to a target protein, or portion thereof, comprising the step of introducing into cells of the patient a vector containing a nucleotide sequence encoding a chimeric immunogen comprising a protein processing signal and the target protein or portion thereof, so that the chimeric immunogen is made within the cells and subsequently processed such that the target protein or portion thereof is presented to the patient's immune system so as to generate a cellular immune response. The chimeric immunogen is said to contain a ubiquitin acceptor which allows for the attachment of ubiquitin by enzymes present in the cytoplasm of the cell, thus targeting the protein for degradation via the ubiquitin-proteasome pathway. The actual results set forth in this patent, however, do not show efficacy under in vivo circumstances that would be encountered in actual therapy, and in fact, the level of immune response generated by these constructs is not sufficient. As shown below, the current invention is a liposomal system useful as a vaccine that does provide efficacy under in vivo circumstances.

### SUMMARY OF THE INVENTION

The present invention provides a liposomal system useful as a vaccine composition comprising:

(a) a peptide having a ubiquitinatable region and an antigenic region,

(b) a pH-sensitive liposomal carrier; and

(c) a bilayer-associated adjuvant, such as MPL.

These compositions are surprisingly effective in inducing an in vivo immune response to the antigen corresponding to the antigenic region.

### BRIEF DESCRIPTION OF THE DRAWINGS

FIG. 1 shows schematically the mechanism of action of the liposomal vaccines of the invention.

FIG. 2 shows a schematic diagram of a peptide component of the liposomes of the invention.

FIG. 3 shows the method of formulation and administration schematically.

FIG. 4 shows the number of IFN-gamma secreting, antigen specific, CD8+ T cells induced as a result of a single vaccination.

FIG. 5 shows intracellular cytokine flow cytometry results also demonstrating the number of antigen specific CD8+ T cells induced as a result of a single vaccination.

FIG. 6 shows difference in tumor incidence as a result of vaccination with liposomes of the invention.

FIG. 7 shows a comparison of the liposomal vaccines of the invention with the same peptide and commercial adjuvants.

FIG. 8 shows effects of liposome encapsulation and bilayer associated adjuvant (MPL) on the effectiveness of liposomal vaccines.

FIG. 9 shows that a ubiquitinatable antigen is required for vaccine potency.

FIG. 10 shows that bilayer-associated CpG oligonucleotide covalently linked to cholesterol is an extremely potent bilayer-associated adjuvant for the invention.

### DETAILED DESCRIPTION OF THE INVENTION

The present invention provides a novel vaccine for the generation of CD8+ T cell responses against epitopes contained in ubiquitinatable long peptide sequences. The invention is based upon the principle that long peptide antigens must first be delivered into the cytoplasm, and then into the proteasome of an antigen presenting cell, in order to obtain efficient processing and presentation of MHC-I epitopes. The vaccine of the invention is a combination of the following 3 components: 1) A peptide antigen containing both ubiquitinatable and antigenic regions, 2) A pH-sensitive liposomal carrier to facilitate uptake and cytoplasmic unloading in APCs, and 3) a bilayer-associated immunostimulant (including, but not limited to, MPL, and CpG oligonucleotides conjugated to membrane-associated components).

The vaccine of the invention, which may be referred to herein as a Ubiquisome, or a Ubiquisome Vaccine, works on the model that long, ubiqutinable peptides can be loaded into antigen-presenting cells for processing and MHC-I presentation, by adjuvanted-liposomal carriers (FIG. 1). This method for eliciting an immune response has not been previously described, and employing an antigen with a proteasome-sorting sequence can circumvent the limitations of the prior art.

By selection of the antigenic region of the peptide, the vaccines of the invention can be used in a variety of applications. These include, without limitation, treatment or prevention/delay of cancer, and treatment and prevention of infections and infectious diseases caused by bacteria, viruses and parasites.

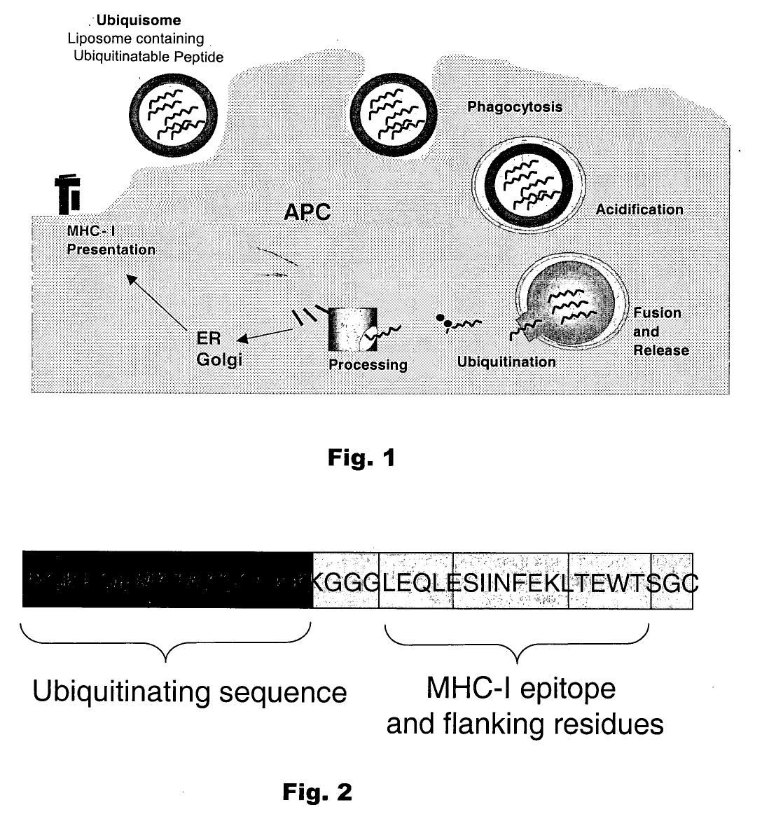

FIG. 2 shows a representation of an exemplary peptide (Seq ID No. 1). It contains a ubiquitinatable sequence (Seq ID No. 2) derived from beta actin protein, a linker (in the figure GGG), an antigenic portion, in the figure the ovalbumin MHC-1 restricted epitope SIINFEKLK (Seq. ID No. 3). The invention, however, is not limited to the specific ubiquitinatable sequences, linker, or the antigenic portion shown.

The ubiquitinatable region of the peptide in the vaccine compositions of the invention may be any ubiquinatable amino acid sequence. Other examples include ubiquitin, as described in U.S. Pat. No. 5,496,721; the E2F ubiquination domain as described in U.S. Pat. No. 6,368,809. These patents are incorporated herein by reference. The antigenic portion of the peptide in the vaccines of the present invention is not limited and may comprise any potentially antigenic epitope or sequence. Specific examples of suitable antigen portions include:

1. Oncofusion protein breakpoint regions

Break point regions are amino acid sequences encoded by the fused regions of chimeric oncofusion genes. Many oncofusion proteins have been identified and associated with different types of malignancies:

| Disease | Oncofusion protein |
|---|---|
| Acute lymphoblastic leukemia | E2A/PBX1 |
| Acute promyelocytic leukemia | PML/RAR |
| Chronic myelogenous leukemia | BCR/ab1 |
| Aveolar rhabdomyosarcoma | PAX3/FKHR |
| Ewing's Sarcoma | EWS/FLI1 |
| Liposarcoma | TLS/CHOP |
| Synovial sarcoma | SYT/SSX |
| Melanoma of soft parts | EWS/ATF1 |
| Sarcoma | ASPL/TFE3 |

Peptides generated from proteolytic processing of oncofusion protein breakpoint regions, if displayed via MHC-I molecules, can be seen by the immune system as neoantigens. This can result in T cell-mediated immunity against oncogene-expressing cells, thus targeting tumor cells while avoiding an autoimmune response. Although these breakpoint regions are ideal targets for immune therapy, they must be processed by the proteasome/MHC I pathway of an antigen presenting cell (APC) in order to elicit an immune response.

There currently exists no method for the delivery of breakpoint antigens into the proteasome/MHC I pathway of APCs. Immunization with DNA encoding the breakpoint region is not an option because of the potential for DNA recombination, leading to malignant cell transformation. The present invention provides a solution to this problem, by delivering long peptides into the MHC-I processing/presentation pathway of APCs in vivo.

2. Any peptide sequence containing a MHC-I-restricted epitope or MHC-I-restricted heteroclitic epitope (epitopes engineered for enhanced binding to MHC or TCR).

Examples of such peptide sequences include, without limitation those described in U.S. Pat. No. 6,287,569 discussed above, namely, there are many tumor antigens that can be recognized by autologous CTL (Boon, T., et al. J. Exp. Med. 183:725-729, 1996; Disis, M. L., et al. Curr. Opin. Immunol. 8:637-642, 1996; Robbins, P. F., et al. Curr. Opin. Immunol. 8:628-636, 1996b). Such antigens are peptide fragments derived from cell proteins that either are restricted to the type of tissue from which the tumor is derived, are mutated during the course of malignant transformation, are aberrantly expressed by the tumor cell, and/or represent “neo” antigens resulting from errors in transcription, RNA processing, translation, and/or protein processing due to a mutation(s) idiosyncratic to the tumor cell. Also, viral antigens are often presented on infected cells and on some tumor cells. There are several examples of antigens that have been found to be recognized by human T cells. These antigens include, but are not restricted to, gp100 (Wolfel, T., et al. Eur. J. Immunol. 24:759-764, 1994; Kawakami, Y., et al. J. Immunol. 154:3961-3968, 1995), MART-1 (MelanA) (Castelli, C., et al. J. Exp. Med. 181:363-368, 1995), tyrosinase (Wolfel, T., et al. Science 269:1281-1284, 1995; Brichard, V. G., et al. Eur. J. Immunol. 26:224-230, 1996; Topalian, S. L., et al. J. Exp. Med. 183:1965-1971, 1996), MAGE-1 (Traversari, C., et al. J. Exp. Med. 176:1453-1457, 1992; van der Bruggen, P., et al. Science 254:1643-1647, 1991), MAGE-3 (Gaugler, B., et al. J. Exp. Med. 179:921-930, 1994), BAGE (Boel, P., et al. Immunity. 2:167-175, 1995), CAGE-1, 2 (Van den Eynde, B., et al. J. Exp. Med. 182:689-698, 1995), N-acetylglucosaminyltransferase-V (Guilloux, Y., et al. J. Exp. Med. 183:1173-1183, 1996), (Robbins, P. F., et al. J. Immunol. 154:5944-5950, 1995), B-catenin (Robbins, P. F., et al. J. Exp. Med. 183:1185-1192, 1996a), MUM-I (Coulie, P. G., et al. Proc. Natl. Acad. Sci. U.S.A. 92:7976-7980, 1995), CDK4 (Kawakami, Y., et al. Proc. Natl. Acad. Sci. U.S.A. 91:6458-6462, 1994), Her-2 (ErbB-2)/neu (Peoples, G. E., et al. Proc. Natl. Acad. Sci. U.S.A. 92:432-436, 1995; Fisk, B., et al. J. Exp. Med. 181:2109-2117, 1995), human papillomavirus-E6, E7 (Ressing, M. E., et al. Cancer Res. 56:582-588, 1996; Alexander, M., et al. Am. J. Obstet. Gynecol. 175:1586-1593, 1996), and MUC-1 (Finn, 0. J., et al. Immunol. Rev. 145:61-89. The sequences of these antigens are set forth in the '569 patent. Utilizing known techniques of recombinant DNA technology, one of ordinary skill in the art could construct peptides which contain these sequences as the antigenic portion for use in the vaccines of the invention.

3. Potentially immunogenic regions of target proteins, which may contain one or more MHC-I-restricted epitopes. Such peptide sequences may be found in virtually any region of any target protein of interest.

The use of long polypeptides, which extend beyond a single epitope, allows for optimum antigen processing and presentation, regardless of the patient's MHC-I haplotype. MHC-II-restricted epitopes can also be included inside of Ubiquisomes to provide CD4+ T cell help.

The liposomal vaccines of the invention are utilized in the treatment or prevention/delay of cancers, infections and/or infectious diseases by introducing the vaccines to an individual, including a human individual, in need of treatment. Introduction of the vaccine in vivo can be carried out by direct injection of the vaccine, for example intramuscular, subcutaneous, subdermal, intravenous, parenteral, and intraperitoneal injection. The amount of liposomal vaccine administered and the frequency of administration will depend on a variety of factors, including the identity of the antigenic species, the tolerance of the individual being treated for the host, and the duration of therapeutic benefit required. Furthermore, in many cases, dosage levels reflect a balancing of detrimental side effects or toxicity with the benefits obtained as a result of the treatment. The determination of dosage amounts and schedules, however, is a matter of standard procedure within the skill in the art. The invention will now be further described with reference to the following, non-limiting examples.

### EXAMPLE 1

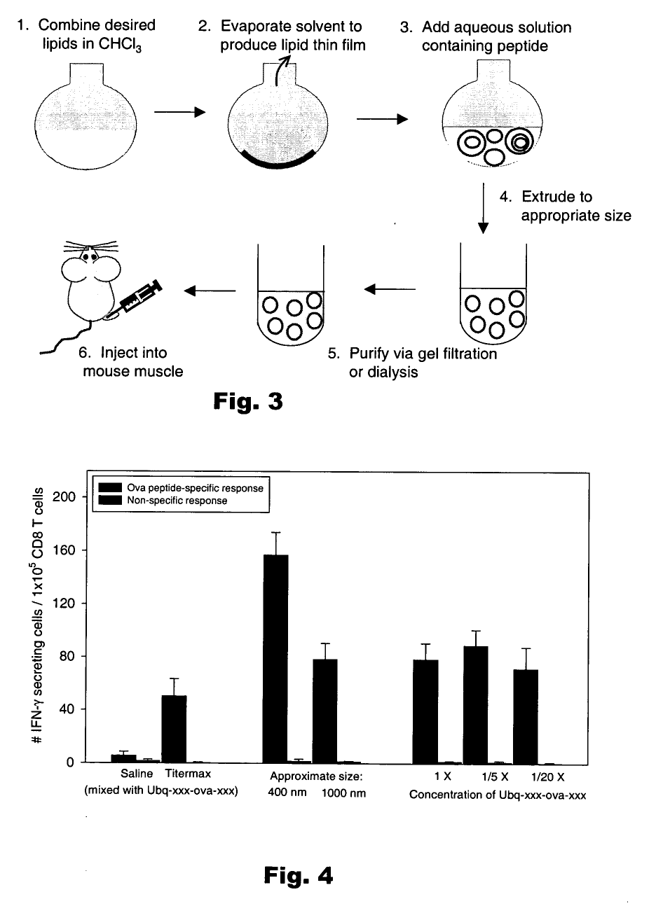

The model ova peptide antigen (FIG. 2, Seq. ID No. 1) was encapsulated in MPL-doped liposomes as outlined in FIG. 3. Liposomes were made according to the thin film rehydration technique. For standard Ubiquisomes, pH-sensitive bilayers were comprised of 70 mole % Palmitoyl-oleoyl-phosphatidylethanolamine (POPE), 30 mole % cholesteryl hemisuccinate (CHEMS) and 0.1-2.0 mole % monophosphoryl Lipid A (MPL). For pH-stable Ubiquisomes, bilayers were comprised of 70 mole % egg yolk phosphatidylcholine, 30 mole % cholesterol, and 2,0 mole % MPL. Lipid thin films were rehydrated by vortexing following addition of dissolved peptide antigen in PBS (pH 8.5). Extrusion of liposomes through polycarbonate membranes produced unilamellar vesicles of the desired size. Unencapsulated peptide was then removed by dialysis against PBS (pH 8.2) or size exclusion chromatography using CL4B resin.

### EXAMPLE 2

An in vitro liposome uptake study was performed. pH-sensitive liposomes encapsulating a quenched concentration (50 mM) of the membrane-impermeable fluorescent dye calcein were added to a fresh splenocyte culture on microscope slides. Slides were rinsed 24 or 48 h later and photographed to visualize uptake of liposomes. Fluorescence images demonstrated dequenching of fluorescence, indicating unloading of liposomal contents into cells. Both punctate and diffuse fluorescence were observed, demonstrating endosomal and cytoplasmic unloading respectively. Light microscope images indicated no liposome uptake by non-adherent cells. Adherent cells from a splenocyte culture demonstrated enhanced uptake of liposomes compared with non-phagocytic cells, suggesting that phagocytosis may be the mechanism of liposomal delivery into APCs.

### Example 3

In order to test the potency of the Ubiquisome vaccine, the liposomes of example 1 were used as a vaccine. For vaccination, mice received a single administration of 100 ml of liposomes containing 2.5 mg POPE and approximately 50 mg peptide antigen. The vaccine was injected bilaterally in mouse tibealis anterior muscle (50 ml per muscle). A single intramuscular injection of the ova ubiquisome vaccine into C57BL/6 mice induced a strong and specific CD8+ T cell response.

For ELISPOT analysis, mice were sacrificed 5 days following vaccination and inguinal and popliteal lymph nodes were harvested. CD8+ T cells from lymph nodes were isolated using the MACS magnetic cell separation technique. 1×10⁵ CD8+ T cells were plated per well in 96 well IP plates. Ova (SIINFEKL, Seq ID No. 3) or irrelevant peptide-pulsed EL4 lymphoma cells were irradiated and plated over CD8+ T cells at density of 10×10⁴ cells/well. Following 20 h incubation and assay development, IFN-g secreting cells were visualized as brown spots on the IP membrane. ELISPOT analysis demonstrates that mice develop a large population of CD8+ T cells recognizing the ova peptide only 5 days following vaccination (FIG. 4).

### EXAMPLE 4

5 days following vaccination in accordance with Example 3, cells from inguinal and popliteal lymph nodes of vaccinated mice were harvested and incubated with a CD8+ T cell-depleted splenocyte culture, either with or without ova peptide (SIINFEKL, Seq. ID No. 3). BFA was added to cultures to prevent Golgi-mediated secretion of IFN-g. After 14 hours incubation, cells were stained with anti-CD8-FITC, then fixed, permeabilized, and stained with anti-IFN-g-PE. Two-color flow cytometry analysis was performed to determine the number of ova-specific CD8+ T cells.

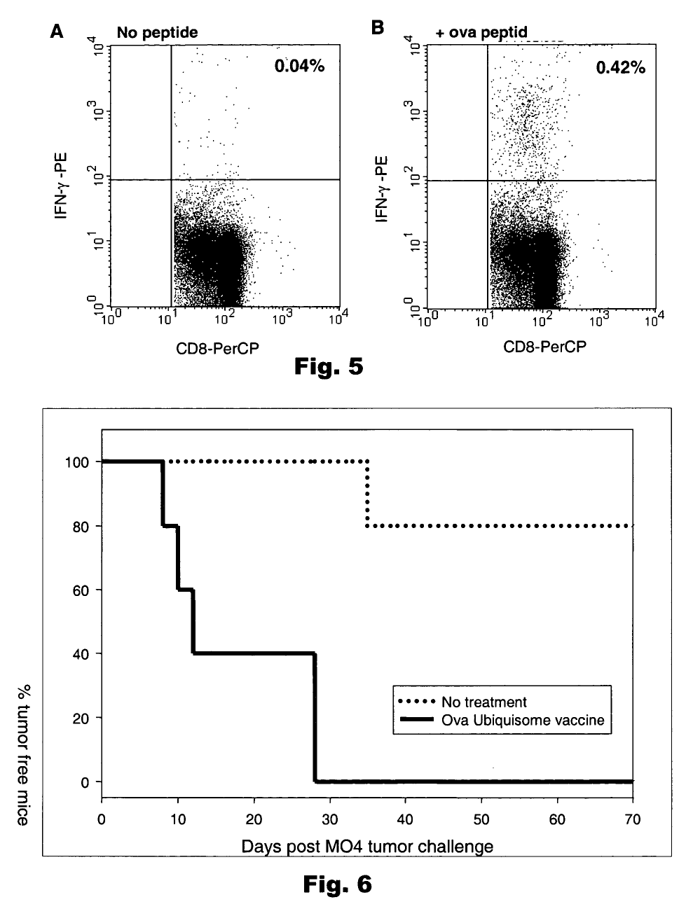

Flow cytometry analysis of intracellular cytokine staining confirms this result, identifying a population of ova-specific T cells equivalent to 0.42% of the total CD8+ T cell population (FIG. 5). While the ELISPOT and flow cytometry analysis determined that a local immune response was mounted, tumor challenge of these mice with B 16 melanoma cells expressing ovalbumin suggests a strong systemic response as well. 4 out of 5 mice vaccinated with Ubiquisomes rejected MO4 tumor challenge, surviving without tumor for over 70 days (FIG. 6).

### EXAMPLE 5

Groups of mice (5 mice/group) were either left untreated or vaccinated with Ubiquisomes (2 mole % MPL, @ 400 nm size) as described in example 3.5 days later, all mice were challenged with an intradermal inoculum of 1×10⁵ MO4 cells (B 16 melanoma cells expressing ovalbumin). Tumor growth was monitored every two days.

### EXAMPLE 6

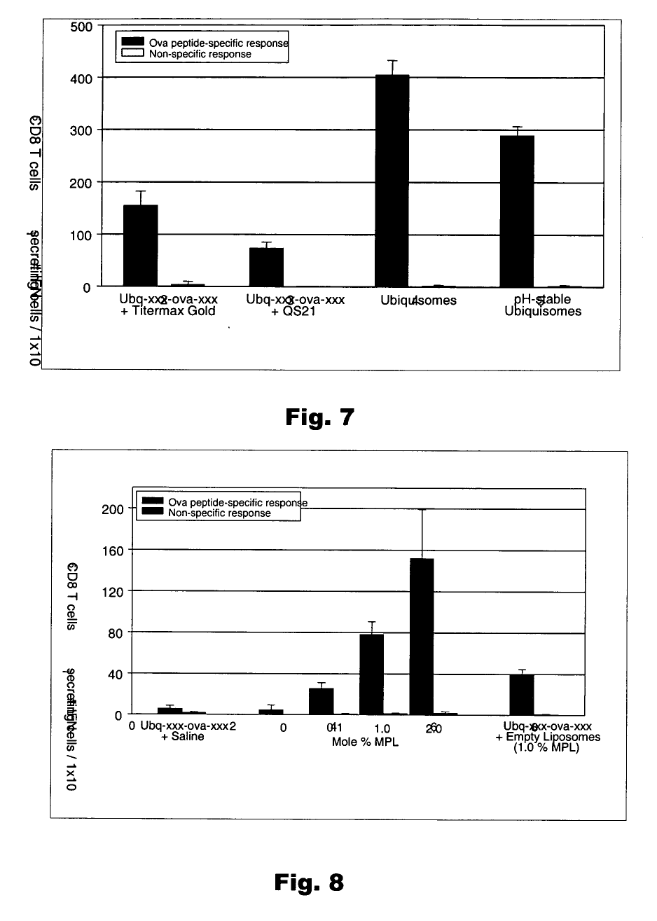

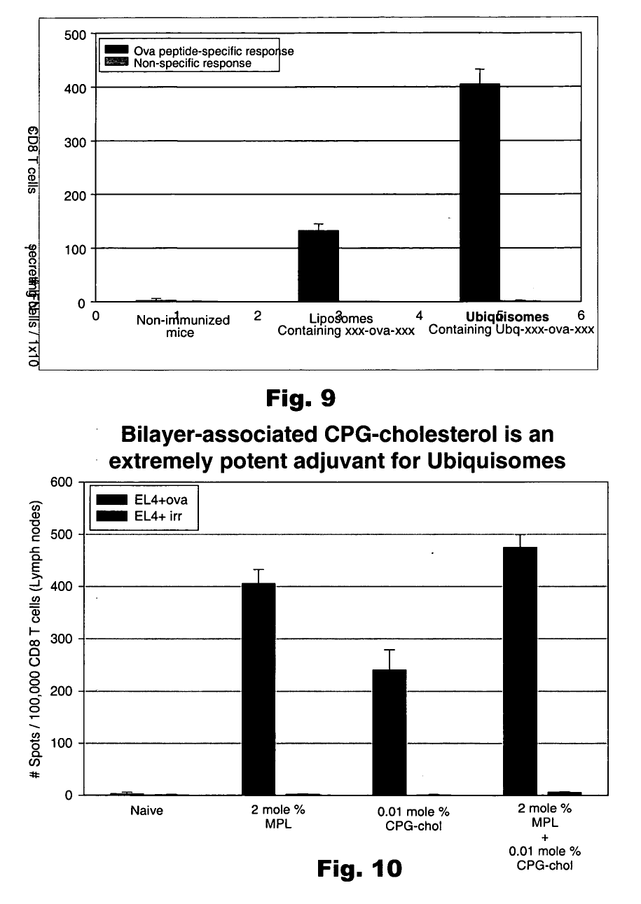

Decreasing the amount of peptide used to make liposomes, even to 1/20 of the original concentration, does not appear to decrease the effectiveness of the vaccine (FIG. 4). This suggests that the liposomes are a highly efficient vehicle for delivery of antigen. When compared with standard peptide adjuvants Titermax Gold and QS21, the MPL-doped liposomal vaccine of the invention elicited 2-5 fold higher numbers of CD8+ T cells against the ova antigen (FIG. 7). This response was dependent on the inclusion of monophosphoryl Lipid A (MPL) as a bilayer-associated immunostimulant, and increasing amounts of MPL elicited proportionally stronger CD8+ T cell responses (FIG. 8). The pH-sensitivity of the liposome enhanced the immune response, however pH stable liposomes also elicited strong CD8+ T cell responses (FIG. 7). Importantly, it was shown that generation of CD8+ T cell immunity requires the presence of a ubiquitinatable peptide sequence, placed N-terminal to the 18 amino acid ovalbumin sequence (FIG. 9). This evidence supports the model of Ubiquisome vaccination involving APC phagocytosis, unloading, and ubiquitin-mediated processing of antigen.

### EXAMPLE 7

Liposomes were made with CPG-cholesterol as a bilayer-associated immunostimulant. Results show that liposomes containing CPG-cholesterol were highly potent when only a small proportion (0.01%) of the molecules in the liposomal bilayer were covalently linked to CPG (FIG. 10). Importantly, CPG-cholesterol, used at only 1/200 the concentration of MPL, achieved T cell responses comparable to MPL. The oligonucleotide CpG motif used was ODN 1826 (sequence TCCATGACGTTCCTGACGTT (Seq. ID No. 4), repeated four times, however other motifs will be relevant for human use.

### EXAMPLE 8

A liposomal vaccine in accordance with the invention was prepared by incorporating the EWS/ATF1 breakpoint having the sequence GGGRGGMGKILKDLSS (Seq. ID No. 5) into a ubiquitinatable peptide having the sequence RGKEQEMATAASSGKKKGGGGGGRGGMGKILKDLSS (Seq ID No. 6). This peptide is incorporated into a liposome as described in Example 1.

### EXAMPLE 9

A liposomal vaccine in accordance with the invention is prepared by incorporating the SYT/SSX breakpoint having the sequence QRPYGYDQIMPKKPAE (Seq. ID No. 7) into a ubiquinatable peptide having the sequence RGKEQEMATAASSGKKKGGGQRPYGYDQIMPKKPAE (Seq ID No. 8). This peptide is incorporated into a liposome as described in Example 1.

### EXAMPLE 10

A liposomal vaccine in accordance with the invention is prepared by incorporating the TLS/CHOP breakpoint having the sequence RGGFNKFGVFKKEVYL (Seq. ID No. 9) into a ubiquinatable peptide (RGKEQEMATAASSGKKKGGGRGGFNKFGVFKKEVYL (Seq. ID No. 10)) which is incorporated into a liposomal system using the procedure of Example 1.

### EXAMPLE 11

A liposomal system in accordance with the invention is prepared by incorporating the ASPL/TFE3 breakpoint having the sequence QQEQERERLPVSGNLL (Seq. ID No. 11) into a ubiquinatable peptide (RGKEQEMATAASSGKKKGGGQQEQERERLPVSGNLL (Seq. ID No. 12) which is incorporated into a liposomal system using the procedure of Example 1.

### EXAMPLE 12

A liposomal system in accordance with the invention is prepared by incorporating the BCR/ABL breakpoint having the sequence IVHSATGFQSSKALQRPVASDFEP (Seq. ID No. 13) into a ubiquinatable peptide (RGKEQEMATAASSGKKKGGGIVHSATGFQSSKALQRPVASDFEP (Seq. ID No. 13) which is incorporated into a liposomal system using the procedure of Example 1.

What is claimed is:

1. A liposomal system comprising:
(a) a peptide having a ubiquitinatable region and an antigenic region,
(b) a pH-sensitive liposomal carrier; and
(c) a bilayer-associated adjuvant.

2. The liposomal system of claim 1, wherein the ubiquitinatable region comprises Seq. ID No. 2

3. The liposomal system of claim 2, wherein the antigenic region is an oncofusion protein breakpoint region.

4. The liposomal system of claim 3, wherein the antigenic region comprises a leukemia-associated oncofusion protein breakpoint region selected from the group consisting of E2A/PBX1, PML/RAR and BCR/abl.

5. The liposomal system of claim 3, wherein the antigenic region comprises a sarcoma-associated oncofusion protein breakpoint region selected from the group consisting of PAX3/FKHR, EWS/FLI1, TLS/CHOP and SYT/SSX, and ASPL/TFE3.

6. The liposomal system of claim 3, wherein the antigenic region comprises a EWS/ATF1 melanoma of soft parts-associated oncofusion protein breakpoint region.

7. The liposomal system of claim 1, wherein the antigenic region is an oncofusion protein breakpoint region.

8. The liposomal system of claim 7, wherein the antigenic region comprises a leukemia-associated oncofusion protein breakpoint region selected from the group consisting of E2A/PBX1, PML/RAR and BCR/abl.

9. The liposomal system of claim 7, wherein the antigenic region comprises a sarcoma-associated oncofusion protein breakpoint region selected from the group consisting of PAX3/FKHR,EWS/FLI1, TLS/CHOP, SYT/SSX, and ASPL/TFE3.

10. The liposomal system of claim 7, wherein the antigenic region comprises a EWS/ATF1 melanoma of soft parts-associated oncofusion protein breakpoint region.

11. The liposomal system of claim 1, wherein the antigenic region comprises a protein or peptide sequence containing a MHC-I-restricted epitope or MHC-I-restricted heteroclitic epitope.

12. The liposomal system of claim 11, wherein the ubiquitinatable region comprises Seq. ID No. 2

13. The liposomal system of claim 1, wherein the bilayer-associated adjuvant is monophosphoryl Lipid A.

14. The liposomal system of claim 1, wherein the bilayer-associated adjuvant is CPG-cholesterol.

15. The liposomal system of claim 14, wherein the CPG-cholesterol comprises Seq. ID No. 4.

16. A method for treating a cancer in a patient, wherein the cancer has a specific antigen associated with it, comprising the step administer to the patient a therapeutically effective amount of a liposome comprising
(a) a peptide having a ubiquitinatable region and an antigenic region,
(b) a pH-sensitive liposomal carrier; and
(c) a bilayer-associated adjuvant,
wherein the antigenic region of the peptide comprises an epitope of the specific antigen associated with the cancer.

17. The method of claim 16, wherein the ubiquitinatable region comprises Seq. ID No. 2

18. The method of claim 17, wherein the antigenic region is an oncofusion protein breakpoint region.

19. The method of claim 18, wherein the cancer is a leukemia, and the antigenic region comprises a leukemia-associated oncofusion protein breakpoint region selected from the group consisting of E2A/PBX1, PML/RAR and BCR/abl.

20. The method of claim 18, wherein the cancer is a sarcoma and the antigenic region comprises a sarcoma-associated oncofusion protein breakpoint region selected from the group consisting of PAX3/FKHR, EWS/FLI1, TLS/CHOP, SYT/SSX, and ASPL/TFE3.

21. The method of claim 18, wherein the cancer is melanoma and the antigenic region comprises a EWS/ATF 1 melanoma of soft parts-associated oncofusion protein breakpoint region.

22. The method of claim 16, wherein the antigenic region is an oncofusion protein breakpoint region.

23. The method of claim 22, wherein the cancer is a leukemia, and the antigenic region comprises a leukemia-associated oncofusion protein breakpoint region selected from the group consisting of E2A/PBX1, PML/RAR and BCR/abl.

23. The method of claim 22, wherein the cancer is a sarcoma and the antigenic region comprises a sarcoma-associated oncofusion protein breakpoint region selected from the group consisting of PAX3/FKHR, EWS/FLI1, TLS/CHOP, SYT/SSX and ASPL/TFE3.

24. The method of claim 22, wherein the cancer is melanoma and the antigenic region comprises a EWS/ATF1 melanoma-associated oncofusion protein breakpoint region.

25. The method of claim 16, wherein the antigenic region comprises a protein or peptide sequence containing a MHC-I-restricted epitope or MHC-I-restricted heteroclitic epitope.

26. The method of claim 25, wherein the ubiquitinatable region comprises Seq. ID No. 2

27. The method of claim 16, wherein the bilayer-associated adjuvant is monophosphoryl Lipid A.

28. The method of claim 16, wherein the bilayer-associated adjuvant is CPG-cholesterol.

29. The method of claim 28, wherein the CPG-cholesterol comprises Seq. ID No. 4.


SEQUENCE LISTING (14 sequences)

SEQ ID NO: 1 - protein, 39 aa, artificial
  fusion with Ova antigen and ubiquinating sequence
RGKEQEMATAASSGKKKGGGLEQLESIINFEKLTEWSGC

SEQ ID NO: 2 - protein, 17 aa, human
RGKEQEMATAASSGKKK

SEQ ID NO: 3 - protein, 8 aa, artificial
  ovalbumin MHC-1 epitope
SIINFEKL

SEQ ID NO: 4 - DNA, 20 bp, artificial
  CpG containing sequence
tccatgacgttcctgacgtt

SEQ ID NO: 5 - protein, 16 aa, artificial
  EEWS/ATF1 breakpoint
GGGRGGMGKILKDLSS

SEQ ID NO: 6 - protein, 36 aa, artificial
  ubiquinatable peptide for EWS/ATF1 breakpoint
RGKEQEMATAASSGKKKGGGGGGRGGMGKILKDLSS

SEQ ID NO: 7 - protein, 16 aa, artificial
  SYT/SSX breakpoint
QRPYGYDQIMPKKPAE

SEQ ID NO: 8 - protein, 36 aa, artificial
  ubiquinatable peptide for SYT/SSX breakpoint
RGKEQEMATAASSGKKKGGGQRPYGYDQIMPKKPAE

SEQ ID NO: 9 - protein, 16 aa, artificial
  TLS/CHOP breakpoint
RGGFNKFGVFKKEVYL

SEQ ID NO: 10 - protein, 36 aa, artificial
  ubuinatable peptide for TLS/CHOP breakpoint
RGKEQEMATAASSGKKKGGGRGGFNKFGVFKKEVYL

SEQ ID NO: 11 - protein, 16 aa, artificial
  ASPL/TFE3 breakpoint
QQEQERERLPVSGNLL

SEQ ID NO: 12 - protein, 36 aa, artificial
  ubiquinatable peptide for ASPL/TFE3 breakpoint
RGKEQEMATAASSGKKKGGGQQEQERERLPVSGNLL

SEQ ID NO: 13 - protein, 24 aa, artificial
  BCR/ABL breakpoint
IVHSATGFQSSKALQRPVASDFEP

SEQ ID NO: 14 - protein, 44 aa, artificial
  ubiquinatable peptide for BCR/ABL breakpoint
RGKEQEMATAASSGKKKGGGIVHSATGFQSSKALQRPVASDFEP